(19) United States
(12) Patent Application Publication
(10) Pub. No.: US 2008/0251672 A1
(43) Pub. Date: Oct. 16, 2008

(54) Support Arrangement

(75) Inventors: Susan Elizabeth Barton, West Yorkshire (GB); Jonathan Philby, West Yorkshire (GB); Mark Robinson, West Yorkshire (GB)

Correspondence Address: MICHAEL BEST & FRIEDRICH LLP, 100 E WISCONSIN AVENUE, Suite 3300, MILWAUKEE, WI, 53202, US

(73) Assignee: University of Bradford, Bradford, West Yorkshire (GB)

(21) Appl. No.: 11/815,590

(22) PCT Filed: Feb. 10, 2006
(86) PCT No.: PCT/GB06/00470

§ 371 Date: Jan. 17, 2008

(30) Foreign Application Priority Data
Feb. 11, 2005 (GB) 0502824.6

(51) Int. Cl. A61B 19/00

(52) U.S. Cl. 248/323

(57) ABSTRACT

The invention is not A support arrangement (2) for medical equipment, the support arrangement (2) being self supporting and having an attachment means (10) for optional attachment to patient support apparatus. The arrangement may include an upwardly extending portion (8) arranged, in use, to be located at a side region of patient support apparatus and an outwardly extending medical support portion (12) arranged, in use, to be located at least partially over the patient support apparatus.

The present invention relates to support arrangements for medical equipment and a method relating thereto.

According to one aspect of the invention there is provided a support arrangement for medical equipment, the support arrangement being self supporting and having an attachment means for optional attachment to patient support apparatus.

The arrangement may include an upwardly extending portion arranged, in use, to be located at a side region of patient support apparatus and an outwardly extending medical support portion arranged, in use, to be located at least partially over the patient support apparatus.

According to another aspect of the present invention a support arrangement for medical equipment includes an upwardly extending portion arranged, in use, to be located at a side region of patient support apparatus and an outwardly extending medical support portion arranged, in use, to be located at least partially over the patient support apparatus.

The support arrangement may be self supporting. Alternatively or additionally the support arrangement may include attachment means for optional attachment to patient support apparatus.

Preferably the attachment means comprises clamping means. The attachment means may have a quick release mechanism. The clamping means may comprise a pair of jaws arranged to engage a part of a patient support. One jaw, or preferably both jaws may be adjustable in the vertical direction relative to the remainder of the apparatus. One or preferably both jaws may be arranged to slide on the arrangement prior to the jaws clamping parts of the patient support apparatus.

The clamping means may be an over centre locking clamp. The clamping means is preferably provided with a fixed jaw and a pivotable jaw. The clamping means may be an eccentric cam type locking clamp.

Preferably a connection device is provided on a bar of the patient support apparatus for receiving the upwardly extending portion. The connection device may comprise a receiving means and a support means for attachment to the bar.

Preferably the support arrangement receiving means comprises a socket for receiving a plug located on the upwardly extending portion. The socket may be provided with a grub screw to hold the socket in firm connection with the plug. Alternatively, the support arrangement receiving means may comprise a plug for receiving a socket located on the upwardly extending portion.

Preferably, the support arrangement receiving means is slidably arranged on the bar. The support means may be provided with rollers to enable it to slide smoothly along the bar. The support means may be provided with means for locking to the bar. The locking means may be one or more quick release clamps.

The patient support apparatus is preferably provided with at least two connection devices. The connection devices may be located such that the suspension unit may be placed in a suitable position for supporting a patients left or right arm or leg.

Preferably the support arrangement includes a base which may be detachable. The base may be connected to the rest of the support arrangement by means of a telescopic arrangement. The base may be connected to the support arrangement by means of a clamp or locking collar, which may be a quick release clamp or collar and may be cam operated. The base may include transporting wheels. The base, when removed, may be stackable with other bases. The base may be removable from the support arrangement by an operative urging the base downwardly. The support arrangement may be height adjustable with respect to the base. The arrangement may include releasable locking means arranged to hold the support arrangement at a selected height with respect to the base. The support arrangement, when not held at the selected height, may be supported on resilient means such as a spring. The resilient means may be arranged to maintain the support arrangement at a distance from the base that is between the limits of height adjustment such as at a mid region between those limits.

Preferably the support arrangement is provided with a safety mechanism such that the attachment means cannot be released unless the base is attached. The safety mechanism may raise the base off the ground when the attachment mechanism is operated.

Preferably the arrangement has a height adjusting mechanism for a medical support portion. The height adjusting mechanism may comprise a geared mechanism such as a rack and pinion mechanism or a worm gear mechanism. A lock may be provided which, when activated, maintains the arrangement at the set height.

The outwardly extending medical support portion may be adjustable in the extent that is arranged to extend over a patient. The medical support portion may comprise an arm which may be mounted by means of a cantilever to the support arrangement. One of the arm or the support arrangement may include a recess with the other including a projection which are arranged to engage with each other to prevent adjustment of the extent that the arm extends over a patient. One of the arm or the support arrangement may include a plurality of projections or recesses whereby the arm can be held in one of a plurality of positions.

A second arm may extend at an angle from the first arm which second arm is arranged, in use, to extend at least partially over the patient support apparatus.

The second arm may extend across and forms a T shape with the first arm. The second arm may have notches on an upper surface for locating medical equipment. The second arm may have guard means at each end to prevent medical equipment from slipping off. The first arm may have means for preventing the arm assembly from being removed from the suspension system.

The patient support apparatus may be a bed.

According to another aspect of the present invention there is provided a modular support arrangement for medical equipment including a base portion, a clamping portion and a medical support portion.

According to another aspect of the present invention a method of operating a medical support arrangement comprises the arrangement being at least initially self supporting and subsequently attaching the arrangement to patient support apparatus.

The method may comprise locating an upwardly extending portion of the arrangement at the side region of the patient support apparatus and causing an outwardly extending medical support portion to be located at least partially over the patient support apparatus.

According to another aspect of the present invention a method of operating a medical equipment support arrangement comprises locating an upwardly extending portion of the equipment at a side region of the patient support apparatus and causing an outwardly extending support portion to extend at least partially over the patient support apparatus.

The method may comprise the apparatus at least initially being self supporting prior to attaching the arrangement to the patient support apparatus.

The method may comprise altering the height of the attachment relative to the remainder of the arrangement prior to attaching the arrangement to the patient support apparatus.

The method may comprise attaching the arrangement to the support apparatus with clamping means. The method may comprise releasing the clamping means via a quick release mechanism.

The method may comprise attaching the arrangement to the patient support apparatus when the patient support apparatus comprises a bed or a trolley or a wheelchair or any combination thereof. The method may also comprise moving the patient support apparatus when the medical support arrangement is attached to the apparatus.

The method may comprise transporting the arrangement by wheels attached to the arrangement. The method may comprise removing a base from the arrangement, such as, for instance, after the arrangement has been attached to the patient support apparatus.

The method may comprise preventing the attachment means from being released until a previously detached base has been attached. The method may comprise raising the base off the ground when the attachment is effected.

The method may comprise adjusting the height of the support apparatus which method may be effected by a geared mechanism.

The method may comprise adjusting the extent that part of the arrangement extends over the patient support.

The method may comprise suspending medical equipment from the support arrangement which equipment is connected to the patient with the equipment extending down such as directly down from a location above the patient.

The method may comprise suspending equipment from the support arrangement which equipment is caused to extend at least partially under a part of the patient and subsequently raising that equipment and thereby the part of the patient that the equipment extends at least partially under.

The present invention includes a method of operating a medical support arrangement when the medical support arrangement is as herein referred to.

The present invention includes any combination of the herein referred to features or limitations.

For a better understanding of the invention, and to show how embodiments of the same may be carried into effect, reference will now be made, by way of example, to the accompanying diagrammatic drawings in which:

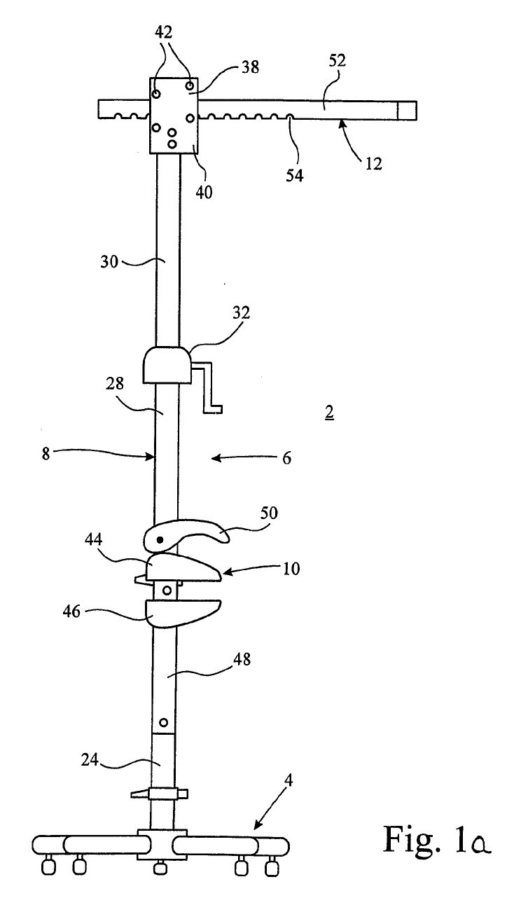

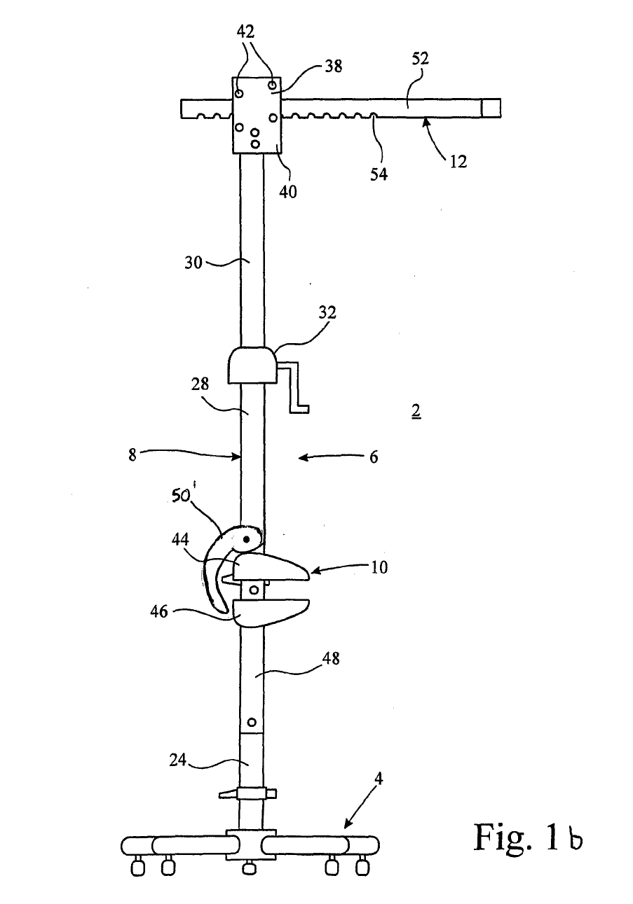

FIGS. 1a and 1b show a side view of a first embodiment of a suspension system;

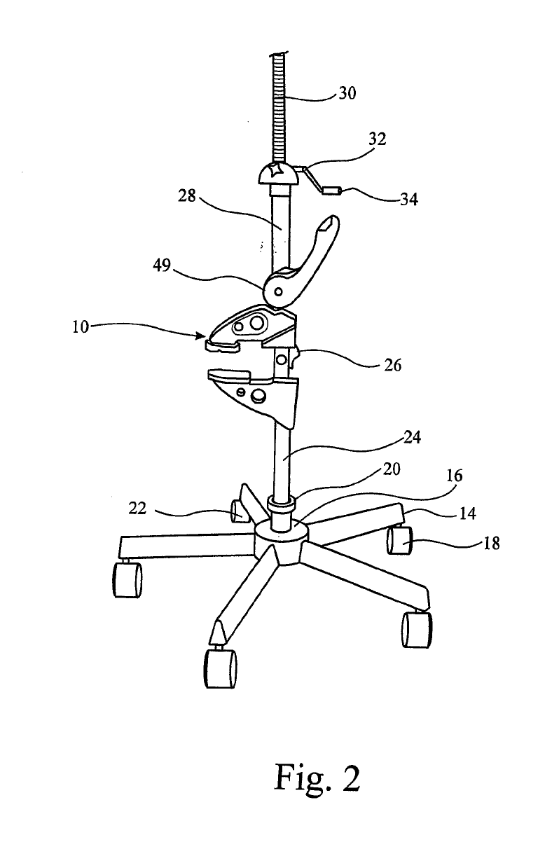

FIG. 2 is a perspective view of a lower part of the suspension system of FIG. 1;

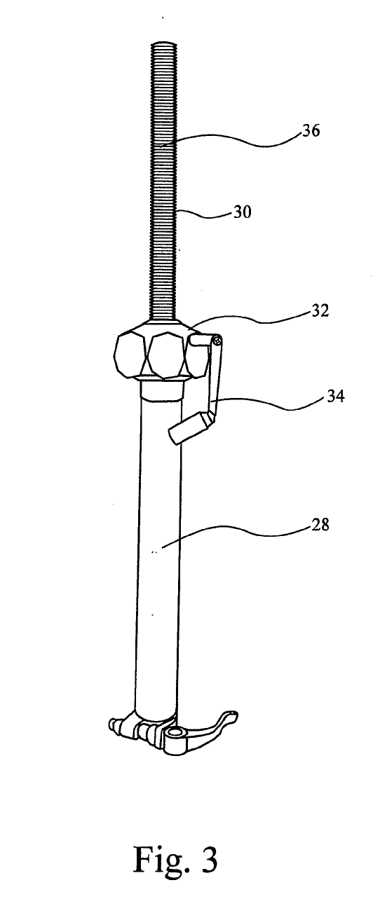

FIG. 3 is a side view of the ratchet mechanism of FIG. 1;

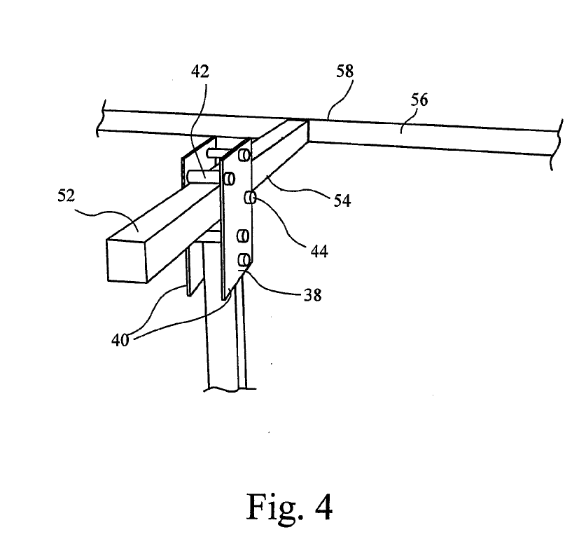

FIG. 4 is a perspective view of an upper part of the suspension system of FIG. 1;

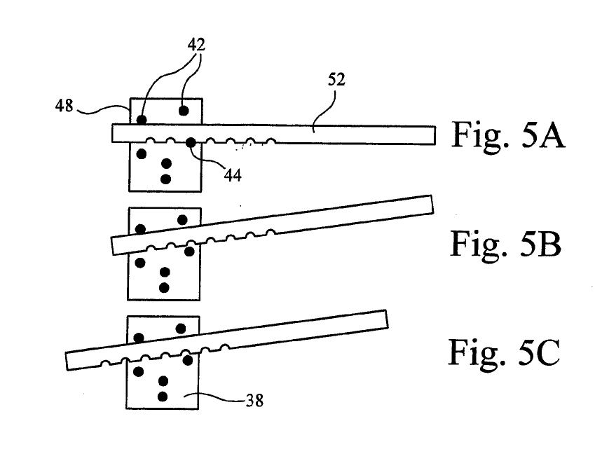

FIGS. 5a-5c are diagrammatic representations of the location of the arm assembly in the support unit;

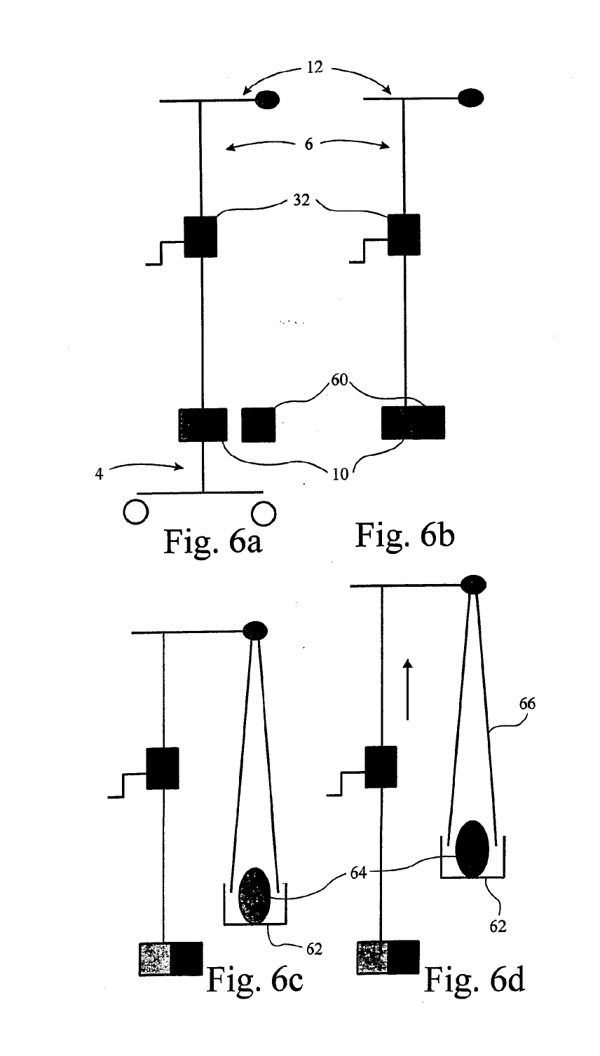

FIGS. 6a-6d are diagrammatic representations of the suspension system of FIG. 1 in use;

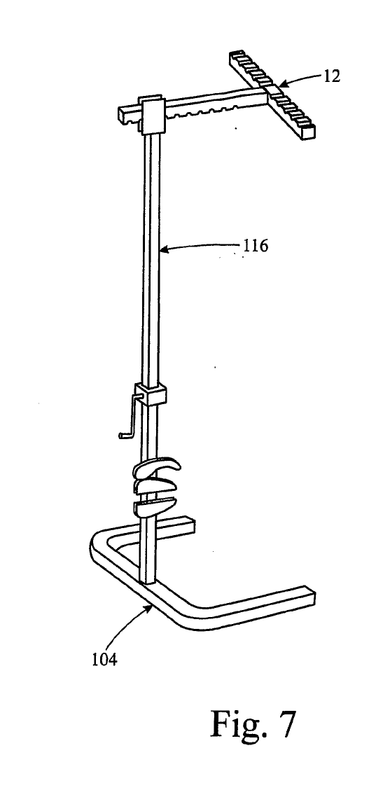

FIG. 7 is a side view of a second embodiment of the suspension system;

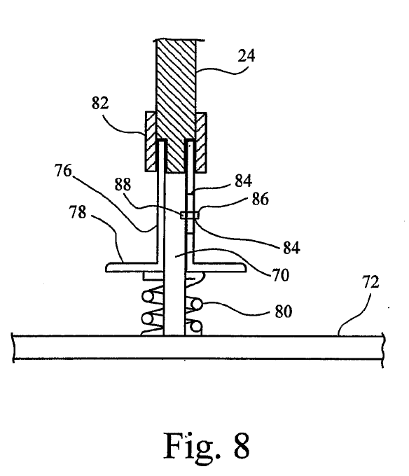

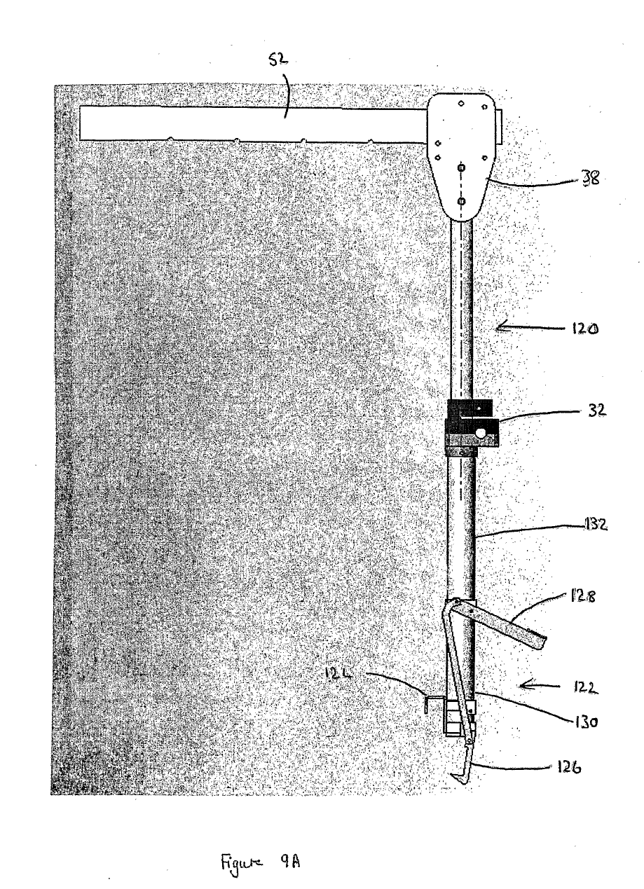

FIG. 8 is a detailed view of the base of either embodiment;

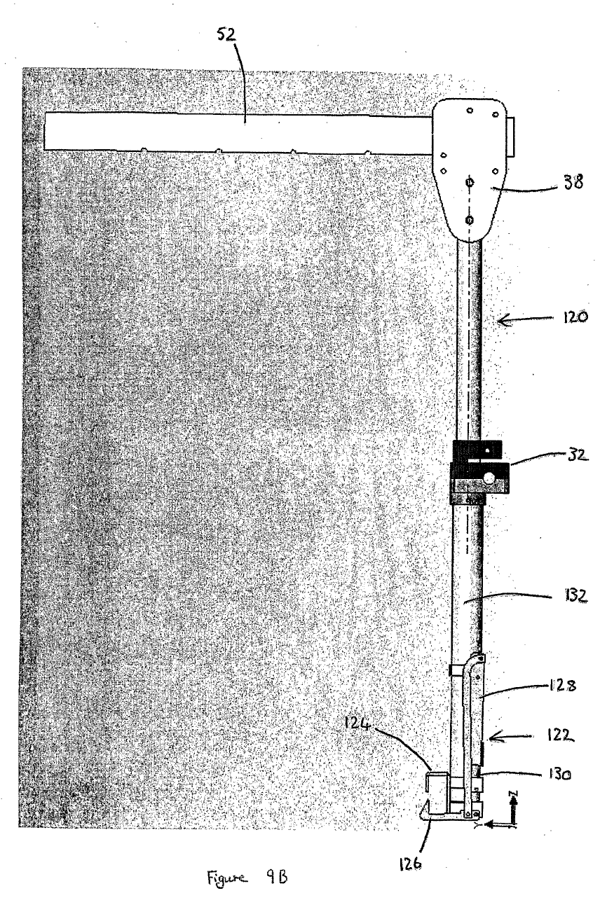

FIGS. 9a and 9b are diagrammatic representations of a further embodiment of the main suspension unit;

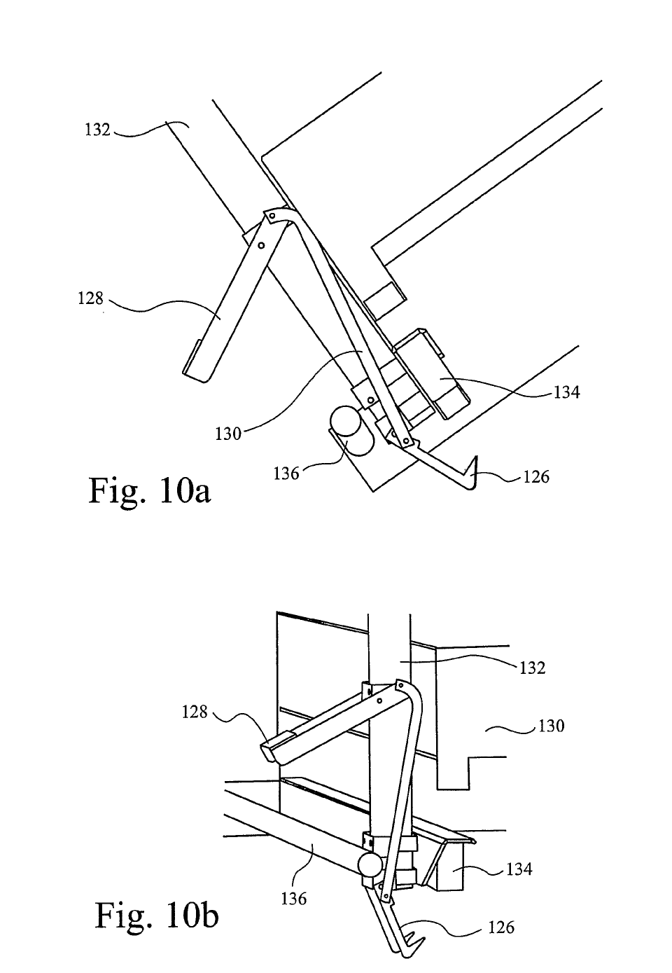

FIGS. 10a-10d are diagrammatic representations of the clamp of FIGS. 9a and 9b;

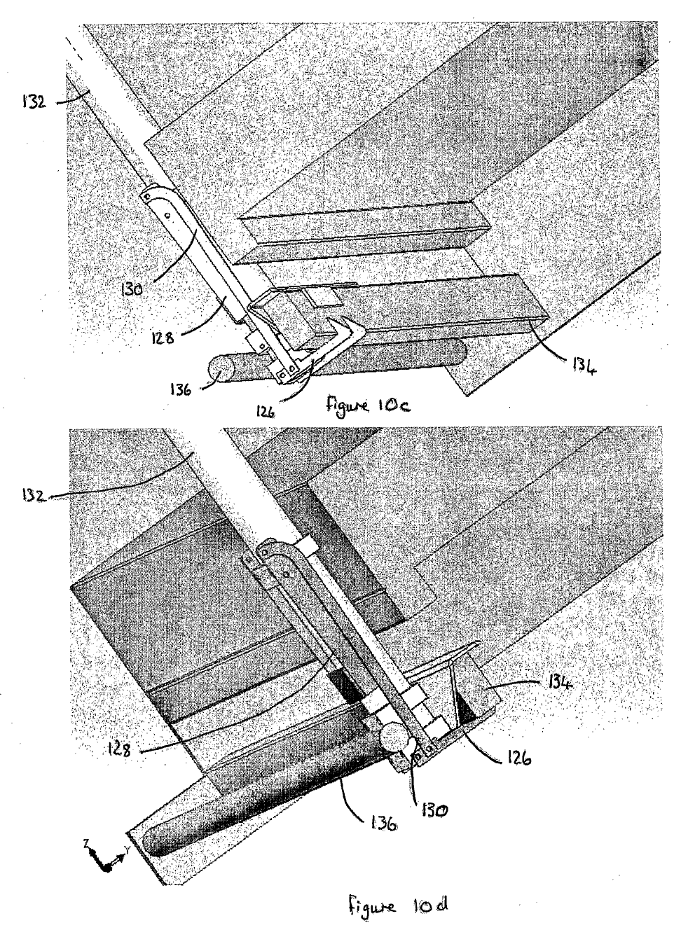

FIGS. 11a and 11b are diagrammatic representations of a further embodiment of the main suspension unit;

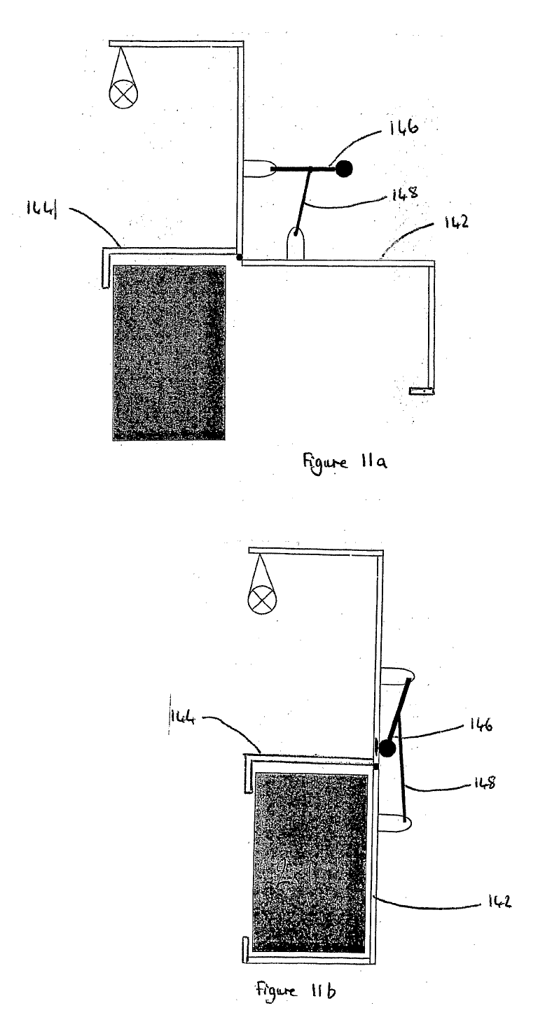

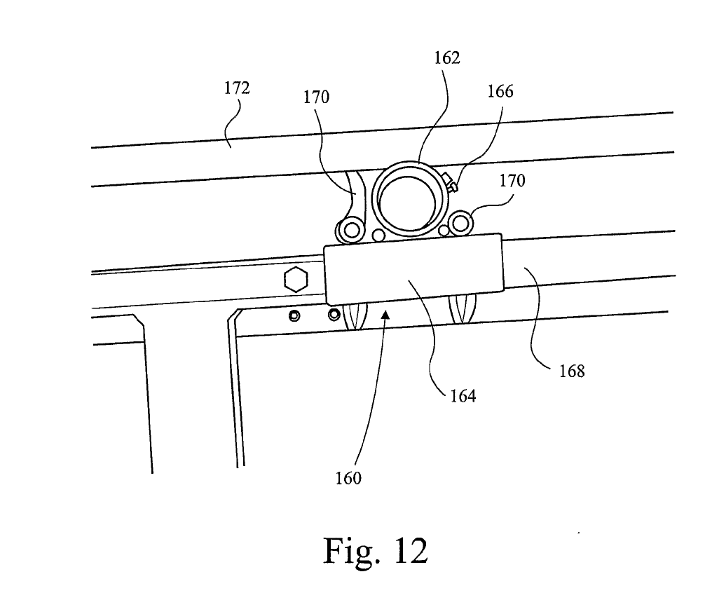

FIG. 12 is a detailed view from below of a connection device for use with a suspension unit according to the present invention;

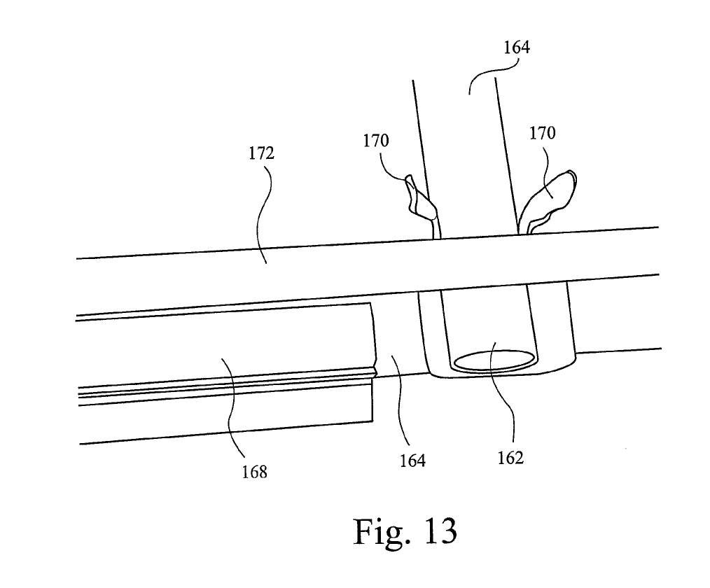

FIG. 13 is a detailed view from below and the side of a connection device for use with a suspension unit according to the present invention;

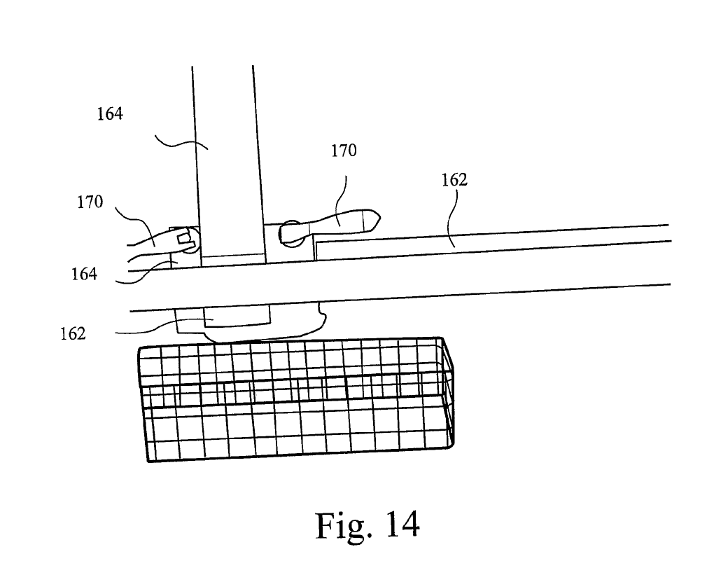

FIG. 14 is a detailed view from the side of a connection device for use with a suspension unit according to the present invention;

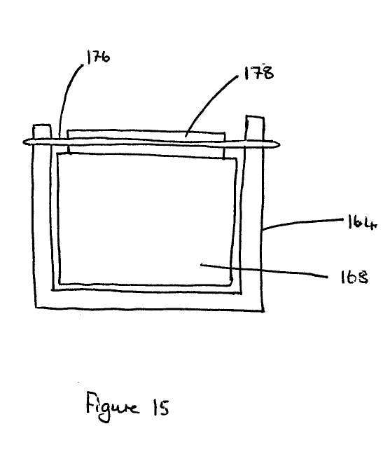

FIG. 15 is a cross sectional view of the carriage of FIGS. 12-14; and

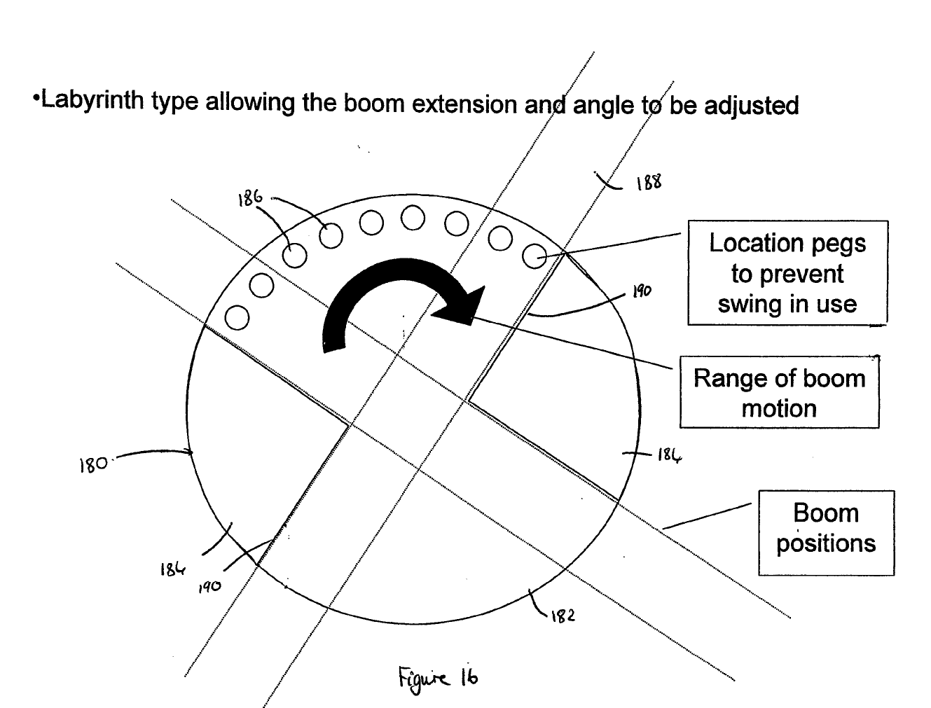

FIG. 16 is a cross sectional view from above of a further embodiment of a support unit; and

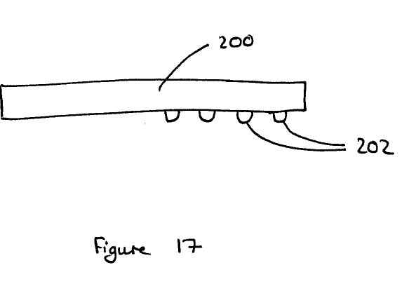

FIG. 17 is a side view of an arm for use in a suspension system according to the present invention.

A suspension system 2 is shown in FIGS. 1a and 1b having a base 4 and a main suspension unit 6. The main suspension unit 6 comprises a support assembly 8, a clamp 10 and an arm assembly 12.

As shown more clearly in FIG. 2, the base 4 comprises five legs 14 extending outwardly from a central hub 16. Each leg has a castor 18 mounted at the outer end thereof. The legs 14 provide a stable platform for the suspension system and may counterbalance loads, such a limb of a patient, suspended from the arm assembly 12.

The castors 18 enable the suspension system 2 to be easily moved around on the floor. A quick release clamp 20 is located on a tubular portion 22 at the top of the hub 16 to enable the base 4 to be removed from a first tubular member 24 and thus the main suspension unit 6. The clamp 20 comprises a collar with a cam operated tightening mechanism.

The support assembly 8 comprises the first tubular member 24 connected telescopically by means of a further quick release clamp 26 to a second tubular member 28. The second tubular member 28 is connected telescopically to a third tubular member 30 by means of a raising or lowering unit 32. The unit 32 comprises a geared rack and pinion mechanism operated by a rotatable handle 34 to raise or lower the third tubular member 30. As shown in FIG. 3, the third tubular member 30 has a series of grooves 36 along a portion of a surface, providing the rack for engagement with the geared mechanism. Alternatively a separate rack may be attached to the third tubular member. The rack and pinion mechanism may be provided with a safety lock to prevent the third tubular member from lowering when under load. The safety lock may be in the form of a pivoting lever which moves freely when the pinion is moved in one direction and locks the pinion when the pinion is moved in the other direction which may comprise a ratchet lever that is biased under gravity to lock the mechanism and which has to be held away from the mechanism to allow lowering.

The third tubular member 30 also has a support unit 38 connected to an upper end by means of two bolts and corresponding nuts. The support unit 38 comprises two spaced plates 40 with four nut and bolts 42 provided between them. The bolts extend through hollow tubes which act as spacing means between the plates 40. Nut and bolts also secure the plates 40 to the member 30.

The clamp 10 is a quick release ratchet clamp for attaching the suspension system 2 to a substantially horizontal bar on a bed. The clamp 10 comprises two jaws 44,46 for gripping either side of the bar. Both jaws 44,46 are held in position under gravity by means of teeth provided along one side of a rail 48. The lower jaw 46 can be moved up or down to the underside of a bar of the bed by tilting the jaw 46 in an anti clockwise direction when viewing FIG. 2 to free the jaw 46 from the notches, sliding the jaw 46 up or down and then releasing the jaw 46. The upper jaw 44 is likewise moved by tilting in a clockwise direction. A pivotable mounted lever 50, 50′ is provided for operating the ratchet mechanism to apply the grip of the jaws 44,46 and has a button for initiating a quick release. The jaws 44, 46 are urged into tight cooperation with a member such as the side of a hospital bed which they are to grip by means of a cam 49 on the lever 50. Lever 50′ when in a clamping position lies substantially parallel with the tubular members 24, 28. The means that lever 50′ is unlikely to become entangled in use with other pieces of equipment or to accidentally catch on a person. If desired, the lever 50, 50′ can be caused to move in an arc along the side of the bed.

The arm assembly 12 is shown in detail in FIG. 4 and comprises a first tubular arm 52 having a series of first notches 54 (seen more clearly in FIG. 1) on a lower side and a second tubular arm 56 disposed across and forming a T shape with the first arm 52 and having a series of second notches 58 on an upper side. One of the first notches 52 engages with a pin 44 on the support unit 40, and a second pin 44 engages with an upper surface of the first arm 52 to hold the first arm 52 in a substantially horizontal operational position, as illustrated in FIG. 5. The arrangement of pins 44 is such that when there is no load on the arm assembly 12 it can easily be removed or adjusted in effective length by tilting the remote end of the arm up, as shown in FIG. 5b and then sliding the arm, as shown in FIG. 5c before lowering the remote end to cause another notch 54 to engage with the pin 44. When in the operational position the cantilever weight of the arm assembly 12 is sufficient to keep the first arm 52 in position. As a load is applied to the second arm 56 the pressure exerted by the first arm 52 on the pins 44 increases and the first arm 52 becomes even more securely held.

The second notches 58 are provided to accommodate/locate medical apparatus, such as straps for slings, drip holder hooks, drip monitors, drip pumps, surgical lights and cameras or any combination thereof. Alternatively, for instance, the camera may be mounted on top of the unit 38 and the lights may be directly attached to the third tubular member 30. The suspension system 2 is particularly suited to supporting slings for limbs of a patient, as the arm assembly can be adjusted to locate straps attached to the slings in the correct position for providing traction or compression to the limb concerned, as will now be described in more detail.

The operation of the system in use for raising the limb of a patient is illustrated in FIGS. 6a-6d. The suspension system is moved next to a bed as shown in FIG. 6a. The suspension system is then clamped to a bar 60 on the bed at the desired extent along the side of the bed including the ends of the sides, if desired. The base 4 is detached from the main suspension unit 6 as shown in FIG. 6b. The clamp 10 ensures that the suspension unit 6 does not move relative to the bed, even when the base 4 is detached. Removing the base means that it does not get in the way of hospital personnel. However, if desired it can remain attached, especially if extra support is required for heavy loads. Generally the suspension system will be expected to support a maximum load of around 30 Kg. In an alternative embodiment (not shown) the base is unable to be detached from the unit until the clamp is on the bed and also the clamp on the bed is unable to be released from the bed until the base is firmly attached to the stand. This may be achieved by the clamp raising the base off the ground when the clamp is applied or the clamp lowering the base when the clamp is released or both.

The position of the first arm 52 is adjusted within the support unit 38 so that the arm assembly 12 extends a desired distance over the bed. One or more slings 62 are located under a limb 64 of a patient and straps 66 of the one or more slings are placed in one or more of the notches 58 of the second arm 56, as shown in FIG. 6c. The arm assembly 12 is raised by means of the geared raising mechanism 32 (which may include a lock or alternatively or additionally, may comprise a rotating, self locking worm gear) engaging with the tubular member so that the limb 64 is raised and suspended in the sling 63, as shown in FIG. 6d. The raising mechanism 32 takes the load of the patients limb, making it unnecessary for personnel to hold the limb.

The suspension system 2 is multipurpose. The arm assembly 12 may be used to support equipment or equipment may be attached to the main suspension unit 6. It will be apparent that equipment such as drips can be suspended from over the bed rather than the drip coming from the side and extending down and towards the patient. Thus sideways forces that may tend to tug the drip out can be avoided. The suspension system 2 may be attached to apparatus such as standard hospital beds, trolleys, operating tables, X-ray tables and wheel chairs. It can be seen that the suspension system can travel with the bed or chair. The suspension system 2 can be adjusted such that it can provide support for medical apparatus at any point over a bed. This is of particular importance when a patient requires a limb, such as an arm or leg, to be supported in a sling. In order to provide the correct compression/traction on the limb the support points for the sling must be in a certain position relative to the limb. The support system is advantageously designed to work in an area 1 m by 2 m in size, and thus the arm assembly 12 should be extendible to provide support at a distance of up to 0.5 m from the support assembly 8.

FIG. 7 shows a second embodiment of the invention. The main difference in the suspension system 102 is that it comprises a base 104 integral with a main suspension unit 116. The base 104 is substantially U-shaped which provides support for loads when freestanding, whilst not protruding from under a bed when clamped thereto.

The bases of the first embodiment may be stacked either horizontally or vertically. The suspension system 102 may likewise be stacked.

As shown in FIG. 8, the base, which may be removable, is adjustable in the up and down direction to alter the height of the clamp 10. A shaft 70 extends up from the removable base 72 and a tubular member 76 extends around and is slidable on the shaft 70. The base of the member 76 has an outwardly extending peripheral flange 78. The flange traps a spring 80 between the flange and the base. The first tubular member 24 is fast with the top of the member 76 by a relockable locking collar 82. The tubular member 76 includes a series of openings 84 through its side wall and a pin can be inserted through any one of these slots in to an opening 88 in order to lock the tubular member with the shaft 70.

With the pin 86 absent, the spring sets the unladen stand at the mid point with respect to the telescopic extent of the tubular member 76 along the shaft 70. To raise or lower the height of the stand a user can use their foot on the flange 78 or can push down on or pull up on the stand. The pin is inserted through the aligned openings 84 and 86 retain the stand at the required height. With the spring holding the stand at the mid adjustment point only a relatively small force is required to alter the height of the stand.

When the stand is clamped to a hospital bed, the collar can be released and the pin removed with an operative urging their foot against the flange 78 to remove the base.

Hospital beds are not always provided with suitable means to which a clamp 10 as described above can be easily attached. This is particularly the case where the bed has sides which can be raised to prevent the patient from falling off the bed and access to the bars of the bed frame is therefore limited.

FIGS. 9 and 10 show an embodiment of a main suspension unit 120 having an over centre locking clamp 122 which is particularly suited to being connected to a bar 134 of a bed when there is limited access. The clamp 122 is provided with an upper jaw 124, a lower jaw 126, a handle 128 and a linkage 130. The handle 128 is linked to and provides control of the lower jaw 126 by means of the linkage 130. When the clamp 122 is in a closed position, the handle 128 lies along a tubular member 132 of the main suspension unit 120. The handle 128 pivots outwardly to a release position in which the lower jaw 126 is pivoted away from the upper jaw in a release position. The suspension unit 120 may be placed in position on the bar 134 of a bed when the handle 128 and the lower jaw 126 are in the release position, as shown in FIGS. 10a and 10b. The lowering of the lower jaw 126 enables the clamp 122 to be passed easily between the bar 134 and a side bar 136 of the bed. The suspension unit 120 is then clamped to the bar 134 by pivoting the handle 128, which in turn raises the lower jaw 126, as shown in FIGS. 10c and 10d.

The main suspension unit 120 may be connected directly to a base as described above when it is not attached to the bar 134 of the bed.

A further embodiment of a main suspension unit 140 is shown in FIG. 11. This arrangement has a lower jaw 142 which is pivoted next to an upper jaw 144. The lever 146 is connected by a linkage 148 to the lower jaw 142 to move the lower jaw 142 between an open position as illustrated in FIG. 11a and a clamped position as illustrated in FIG. 11b.

The main suspension unit 140 may be connected directly to a base as described above when it is not attached to a bar of a bed.

In a further embodiment of the invention, the bed may be provided with a connection device for receiving the suspension unit. FIGS. 9 to 11 show an example of such a connection device attached to a bed. This arrangement is particularly useful when space around the bed frame is limited.

Referring to FIG. 12, the connection device 160 comprises a socket 162 and a carriage 164. The socket 162 is in the form of a closed cylinder for receiving a cylindrical end 174 of a suspension unit. The socket 162 is provided with a grub screw 166 for clamping the end 174 of the suspension unit firmly in the socket 162.

The carriage 164 supports the socket 162 on a bar 168 such that the suspension unit is maintained in an upright position. The carriage 164 is slidable on parallel rollers 178 along the bar 168, and is locked in the desired position for use by means of two quick release clamps 170. As is shown in FIG. 15, the carriage 164 is generally U-shaped in cross section and is provided with two support pins 176 (only one shown) provided with the rollers 178.

The size of the socket 162 is determined by the space available around the bed. As can be seen in FIG. 12, the socket 162 is located between the bar 168 forming part of the bed frame and a bar 172 forming part of a raisable side of the bed.

The bed is preferably provided with four connection devices. In this case the connection devices may be located such that the suspension unit may be placed in a suitable position for supporting a patients left or right arm or leg.

The socket of the main suspension unit 160 may be connected directly to a base as described above when it is not attached to a bar of a bed.

A further embodiment of a support unit 180 is shown in FIG. 16. The support unit 180 comprises two circular plates 182 (only one shown) having a pair of spacers 184 located therebetween. The plates 182 are provided with a plurality of holes 186 located along two diametrically opposed portions of their circumference. In use an arm 188 is placed through the gap created by the spacers 184 and held in position either between four pins each located in a hole 186 or by two pins each located in a holes 186 and an edge 190 of each spacer 184.

A further embodiment of an arm 200 which may be used in the suspension system of the present invention is shown in FIG. 17. This arm is provided with a plurality of hooks 202 to which various medical devices can be attached as required. The arm 200 projects from one side only from the support.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A support arrangement for medical equipment, the support arrangement including an upwardly extending portion arranged, in use, to be located at a side region of patient support apparatus and an outwardly extending medical support portion arranged, in use, to be located at least partially over the patient support apparatus.

2. The support arrangement of claim 1, wherein the support arrangement is self supporting.

3-11. (canceled)

12. The support arrangement of claim 1, wherein a connection device is arranged to be provided on a bar of the patient support apparatus for receiving the upwardly extending portion.

13. The support arrangement of claim 12, wherein the connection device comprises a receiving means and a support means for attachment to the bar.

14. The support arrangement of claim 13, wherein the receiving means comprises a socket on one of the bar or upwardly extending portion for receiving a plug located on the other of the bar or upwardly extending portion.

15. The support arrangement of claim 14, wherein the socket is provided with a grub screw to hold the socket in firm connection with the plug.

16. The support arrangement of claim 14, wherein the support means is slidably arranged on the bar.

17. The support arrangement of claim 16, wherein the support means is provided with rollers to enable it to slide along the bar.

18. The support arrangement of claim 14, wherein the support means is provided with means for locking to the bar.

19. The support arrangement of claim 18, wherein the locking means comprises one or more quick release clamps.

20. The support arrangement of claim 12, wherein the patient support apparatus is provided with two or more and preferably four connection devices.

21. The support arrangement of claim 20, wherein the connection devices are located such that the upwardly extending portion and outwardly extending portion are placed in a suitable position for supporting a patient's left or right arm or leg.

22. The support arrangement of claim 1, wherein the support arrangement includes a base which is detachable.

23. The support arrangement of claim 22, wherein the base is connected to the rest of the support arrangement by means of a telescopic arrangement.

24. The support arrangement of claim 22, wherein the base may be connected to the support arrangement by means of a clamp.

25. The support arrangement of claim 22, wherein the base may be connected to the support arrangement by means of a locking collar.

26. The support arrangement of claim 24, wherein the clamp or collar is quick release.

27. The support arrangement of claim 24, wherein the clamp or collar is cam operated.

28. The support arrangement of claim 22, wherein the base includes transporting wheels.

29. The support arrangement of claim 22, wherein the base, when removed, is stackable with other bases.

30. The support arrangement of claim 22, wherein the base is removable from the support arrangement by an operative urging the base downwardly.

31-36. (canceled)

37. The support arrangement of claim 1, wherein the arrangement has a height adjusting mechanism for a medical support portion.

38. The support arrangement of claim 37, wherein the height adjusting mechanism comprises a geared mechanism such as a rack and pinion mechanism or a worm gear mechanism.

39. The support arrangement of claim 37, wherein a lock is provided which, when activated, maintains the arrangement at the set height.

40-73. (canceled)
